(19) United States
(12) Patent Application Publication
(10) Pub. No.: US 2019/0216762 A1
(43) Pub. Date: Jul. 18, 2019

(54) VEGETARIAN COMPOSITION CONTAINING UNSATURATED FATTY ACIDS

(71) Applicant: TAIWAN INDIGENA BOTANICA CO., LTD., Taipei City (TW)

(72) Inventors: CHING-HUA SU, Taipei City (TW); CHENG-JENG TAI, Taipei City (TW); ANG-HSI LIN, Taipei City (TW); YEU-CHING SHI, New Taipei City (TW)

(21) Appl. No.: 16/361,711

(22) Filed: Mar. 22, 2019

Related U.S. Application Data
(63) Continuation of application No. 15/429,158, filed on Feb. 10, 2017.

(51) Int. Cl. A61K 31/202; A23D 9/04; A23D 9/013; A61K 31/355; A61K 36/55; A61K 36/47; A61K 36/02

(52) U.S. Cl. CPC A61K 31/202; A23D 9/04; A23D 9/013; A61K 36/02; A61K 36/55; A61K 36/47; A61K 31/355

(57) ABSTRACT

Disclosures of the present invention describe an unsaturated-fatty-acid-containing vegetarian composition, wherein the said composition is composed by at least one vegetable oil and at least one algal oil, and rich in Omega-3, Omega-6 and Omega-9. The present invention particularly design the constituting ratio of Omega-3, Omega-6, and Omega-9 in order to facilitate the content levels of Omega-3 and Omega-6 in the body of a vegetarian administering the vegetarian composition of the present invention reach an ideal balance state. Moreover, it is worth introducing that, this vegetarian composition can be accommodated by a vegetable capsule shell, so as to become a capsule type of dietary supplement. Therefore, it is a great relief for vegetarians to administer the vegetarian composition containing unsaturated fatty acids because the vegetarian composition is made of vegan materials.

### CROSS-REFERENCE TO RELATED APPLICATIONS

This application is a continuation application (CA) of co-pending U.S. patent application Ser. No. 15/429,158 filed on Feb. 10, 2017 and published as U.S. Patent Application Publication No. 2018/0228755A1 on Aug. 16, 2018. The above referenced application, and each document cited or referenced in the above referenced application, are hereby incorporated herein by reference.

### FIELD OF THE INVENTION

The present invention relates to vegetable products rich in Omega-3 and Omega-6, and more particularly to a vegetarian composition containing unsaturated fatty acids.

### BACKGROUND

Omega-3 and Omega-6, also respectively called n-3 and n-6 fatty acid, are two essential fatty acids for human. Deficiencies in these fatty acids lead to a host of symptoms and disorders including: abnormalities in the liver and the kidneys, reduced growth rates, decreased immune function, depression, and dryness of the skin.

Omega-3 fatty acid is one kind of polyunsaturated fatty acid (PUFA). The three types of omega-3 fatty acids involved in human physiology are α-linolenic acid (ALA), eicosapentaenoic acid (EPA), and docosahexaenoic acid (DHA). As doctors or bioengineers skilled in health food developing and manufacturing technology field know, human body is unable to synthesize omega-3 fatty acids, but can obtain through some specific diets, such as bonito, salmon, tuna, mackerel, or herring. On the other hand, human can also obtain the short-chain omega-3 fatty acid ALA from some vegetable oils including spinach oil, basil oil, linseed oil, rapeseed oil, and soybean oil, and then transform the ALA to the more important long-chain omega-3 fatty acids, i.e., EPA and DHA. However, due to low conversion efficiency, it is recommended to directly obtain EPA and DHA trough the aforesaid specific diets instead of the vegetable oils containing ALA.

Omega-6 (Linoleic acid, LA) is also a polyunsaturated fatty acid (PUFA), which can be obtained from some specific vegetable oils, including: soybean oil, corn oil, sunflower oil, safflower oil, cottonseed oil, and sesame oil. Gama-linolenic acid (GLA) is converted to an intermediate in the metabolism of LA by delta-6-desaturase in human body. However this converting reaction is very slow and further restricted by a verity of factors, such as alcohol use, stress, smoking, saturated and trans-fatty acid intake, deficiencies of zinc, cancers, diabetes, and specific immune function. So that, it is able to understand that, there has little amount of bio-activity GLA can be converted from the LA in a man's body even if the man obtains sufficient amount of Omega-6 from the specific vegetable oils.

It is worth demonstrating that, because the vegetarianism is currently in vogue, nutritionists and doctors worry about the vegetarians may suffer from certain disease due to insufficiency of EPA and DHA, such as hyperlipidemia, cardiovascular disease, hemorrhagic stroke, or hypertension. Although the linoleic acid (i.e., Omega-6) obtained from specific vegetable oils can be converted to EPA or DHA in the vegetarian's body, but it is impossible for the Omega-6 level and the Omega-3 level in the vegetarian's body to have a better ratio ranging from 1:1 to 1:5.

Inventors of the present invention have found some microalgaes like Porphyridium cruentum, Nannochloropsis sp., and Phaeodactylum tricornutum contain abundant of EPA. Moreover, the inventors of the present invention have also found some microalgaes are rich in DHA, such as Crypthecodinium cohnii and Chroomonas salin. So that, the inventors of the present application have made great efforts to make inventive research thereon and eventually provided a vegetarian composition, wherein the said vegetarian composition is composed by at least one vegetable oil and at least one algal oil, and rich in containing unsaturated fatty acids including Omega-3, Omega-6 and Omega-9.

### SUMMARY

The primary objective of the present invention is to provide a vegetarian composition containing unsaturated fatty acids. Differing from commercial dietary supplements of EPA and DHA are made by using abyssal fishes as primary raw materials, the present invention provides an unsaturated-fatty-acid-containing vegetarian composition, wherein the said composition is composed by at least one vegetable oil and at least one algal oil, and rich in Omega-3, Omega-6 and Omega-9. The present invention particularly design the constituting ratio of Omega-3, Omega-6, and Omega-9 in order to facilitate the content levels of Omega-3 and Omega-6 in the body of a vegetarian administering the vegetarian composition of the present invention reach an ideal balance state. Moreover, it is worth introducing that, this vegetarian composition can be accommodated by a vegetable capsule shell, so as to become a capsule type of dietary supplement. Therefore, it is a great relief for vegetarians to administer the vegetarian composition containing unsaturated fatty acids because the vegetarian composition is made of vegan materials.

In order to achieve the primary objective of the present invention, the inventor of the present invention provides an embodiment for the vegetarian composition containing unsaturated fatty acids, comprising at least one vegetable oil and at least one algal oil; wherein the said unsaturated fatty acids contained by the vegetarian composition are an Omega-3 fatty acid, an Omega-6 fatty acid and an Omega-9 fatty acid; moreover, and the Omega-3 fatty acid has a first content percent of 45-55%, the Omega-6 fatty acid has a second content percent of 5-15%, and the Omega-9 fatty acid has a third content percent of 10-20%.

In the embodiment of the vegetarian composition, the at least one vegetable oil and at least one algal oil compose the vegetarian composition by a first weight percent of 50-60 wt % and a second weight percent of 35-50 wt %.

The embodiment of the vegetarian composition, further comprising a vitamin, and the at least one vegetable oil, the at least one algal oil and the vitamin compose the vegetarian composition by a first weight percent of 50-60 wt %, a second weight percent of 35-50 wt % and a third weight percent of 0-5 wt %.

In the embodiment of the vegetarian composition, the vegetable oil is selected from the group consisting of: Sacha inchi oil, linseed oil, Chia seed oil, rapeseed oil, combination of the aforesaid two or more vegetable oils.

In the embodiment of the vegetarian composition, the algal oil is selected from the group consisting of: algal oil extracted from Porphyridium cruentum, algal oil extracted from Nannochloropsis sp., algal oil extracted from Phaeodactylum tricornutum, algal oil extracted from Crypthecodinium cohnii, algal oil extracted from Chroomonas salina, mixture of the aforesaid two or more algal oils.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a side view of a capsule accommodating a vegetarian composition containing unsaturated fatty acids according to the present invention;

FIG. 2 shows an exploded view of the capsule;

FIG. 3 shows an exploded view of a combination type capsule housing;

FIG. 4 shows a cross-sectional side view of the combination type capsule housing.

### DETAILED DESCRIPTION

To more clearly describe a vegetarian composition containing unsaturated fatty acids according to the present invention, embodiments of the present invention will be described in detail with reference to the attached drawings hereinafter.

In the present invention, the first embodiment of the vegetarian composition containing unsaturated fatty acids mainly comprises at least one vegetable oil and at least one algal oil, and the said unsaturated fatty acids contained by the vegetarian composition are an Omega-3 fatty acid, an Omega-6 fatty acid and an Omega-9 fatty acid. In order to facilitate the content levels of Omega-3 and Omega-6 in the body of a vegetarian administering the vegetarian composition reach an ideal balance state, it needs to properly modulate the mixing weight percent of the vegetable oils and the algal oils, so as to make the Omega-3 fatty acid, the Omega-6 fatty acid and the Omega-9 fatty acid have a first content percent of 45-55%, a second content percent of 5-15%, and a third content percent of 10-20%, respectively.

In the first embodiment, the said vegetarian composition is composed of the at least one vegetable oil by a first weight percent of 50-60 wt % and the at least one algal oil by a second weight percent of 35-50 wt %. It is worth explaining that, this vegetarian composition can be further processed to a powder type, tablet type, or a capsule type dietary supplement of EPA/DHA only supplied to vegetarians. In addition, the vegetarian composition can also be constituted by at least one vegetable oil, at least one algal oil and a vitamin E, wherein the vitamin E is extracted from vegetable oils, walnuts, sunflower seeds, almonds, wheat germ, embryo rice, brown rice, whole grains, or green leafy vegetables, and has a third weight percent in a range from 0 wt % to 5 wt %.

Herein, it needs to further explain that, the vegetable oil can be a Sacha inchi oil, a linseed oil, a Chia seed oil, a rapeseed oil, or a combination of the aforesaid two or more vegetable oils. On the other hand, the algal oil can be an algal oil extracted from Porphyridium cruentum, an algal oil extracted from Nannochloropsis sp., an algal oil extracted from Phaeodactylum tricornutum, an algal oil extracted from Crypthecodinium cohnii, an algal oil extracted from Chroomonas salina, or a mixture of the aforesaid two or more algal oils.

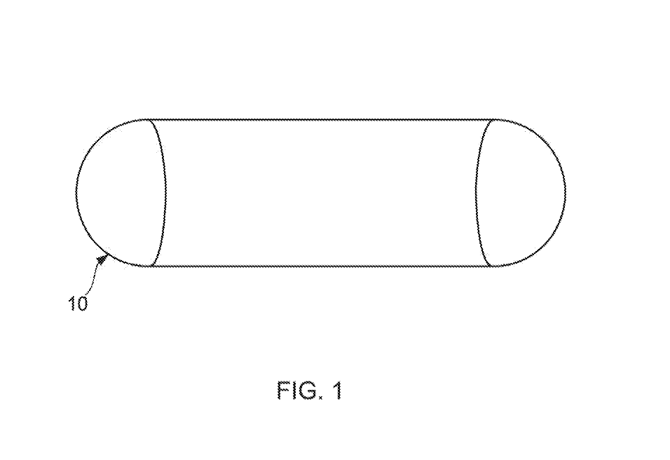

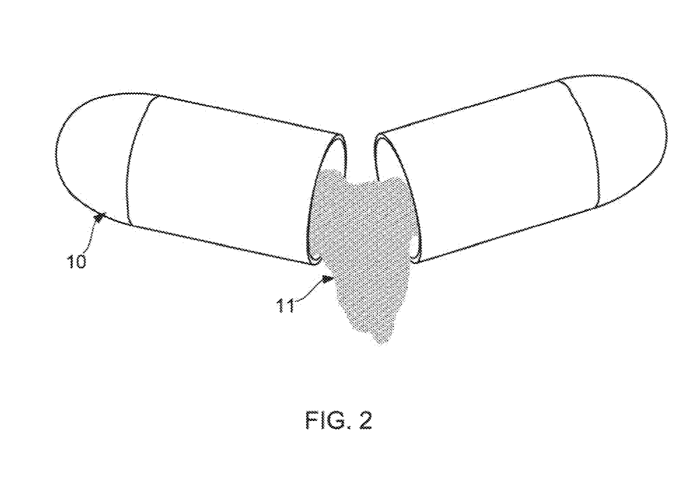

Please refer to FIG. 1, which illustrates a side view of a capsule accommodating a vegetarian composition containing unsaturated fatty acids according to the present invention. As FIG. 1 shows, the second embodiment of the vegetarian composition is a capsule. Moreover, please simultaneously refer to FIG. 2, which shows an exploded view of the capsule. From FIG. 1 and FIG. 2, it can find that, the second embodiment of the vegetarian composition consists of: a capsule housing 10 and a functional essence core 11, wherein the weight percent of the capsule housing 10 in the capsule of the vegetarian composition is in a range from 10 wt % to 35 wt %.

Of course, the capsule housing 10 is also made of at least one vegetarian material. For example, the capsule housing 10 is a vegetable capsule shell made by hydroxypropyl methylcellulose (HPMC) extracted from pines or cottons. On the other hand, the capsule housing 10 can also be made by using the HPMC and carrageenan as raw materials.

In the present invention, the weight percent of the functional essence core 11 in the capsule of the vegetarian composition is particularly designed to in a range from 65 wt % to 90 wt %. As FIG. 2 shows, the functional essence core is a liquid vegetarian composition mainly comprises at least one vegetable oil and at least one algal oil, and contains multi unsaturated fatty acids of Omega-3 fatty acid, Omega-6 fatty acid and Omega-9 fatty acid. The vegetable oil can be a Sacha inchi oil, a linseed oil, a Chia seed oil, a rapeseed oil, or a combination of the aforesaid two or more vegetable oils. On the other hand, the algal oil can be an algal oil extracted from Porphyridium cruentum, an algal oil extracted from Nannochloropsis sp., an algal oil extracted from Phaeodactylum tricornutum, an algal oil extracted from Crypthecodinium cohnii, an algal oil extracted from Chroomonas salina, or a mixture of the aforesaid two or more algal oils.

In the functional essence core 11, the Omega-3 fatty acid, the Omega-6 fatty acid and the Omega-9 fatty acid respectively have a first content percent of 45-55%, a second content percent of 5-15%, and a third content percent of 10-20%. By the particular design of different content percent between the Omega-3, the Omega-6 and the Omega-9 fatty acids, the content levels of Omega-3 and Omega-6 in the body of a vegetarian administering the capsule of the vegetarian composition would reach an ideal balance state.

As doctors or bioengineers know, commercial functional food (health food) or dietary supplement are commonly composed of a primary nutrient ingredient and at least one nutrition strengthening ingredient. So that, the second embodiment of the vegetarian composition also comprises a nutrition strengthening ingredient, which is vitamin E extracted from vegetable oils, walnuts, sunflower seeds, almonds, wheat germ, embryo rice, brown rice, whole grains, or green leafy vegetables.

Continuously, please refer to following Table (1) and Table (2). From the two tables, it is able to know the best values of the first content percent, the second content percent and the third content percent are 50.24%, 11.86% and 14.97%, respectively. Moreover, it can also know that the ALA contained by the Omega-3 fatty acid is contributed by Sacha inchi oil and linseed oil, and the EPA and the DHA contained by the Omega-3 fatty acid are contributed by the algal oil. It is worth explaining that, the said algal oil is a mixture of a first algal oil extracted from Schizochytrium sp. and a second algal oil extracted from Crypthecodinium cohnii.

TABLE (1)

| Functional | | Omega 3 | | | | | |
|---|---|---|---|---|---|---|---|
| essence core | | ALA | | EPA | | DHA | |
| | Content | content | Content | content | Content | content | Content |
| Ingredient | (mg) | percent | (mg) | percent | (mg) | percent | (mg) |
| Sacha inchi | 77.0 | 48.00% | 36.96 | — | — | — | — |
| oil | | | | | | | |
| Linseed | 215.0 | 61.50% | 132.23 | — | — | — | — |
| oil | | | | | | | |
| Algal oil | 200.0 | — | — | 1.0% | 2 | 40.0% | 80.0 |
| Vitamin E | 8.0 | — | — | — | — | — | — |
| Sumation | 500.0 | — | 169.19 | — | 2.0 | | 80.0 |
| Content | — | — | 33.84% | — | 0.4% | — | 16.0% |
| percent | | | | | | | |

TABLE 2

| | | Omega 6 | | Omega 9 | |
|---|---|---|---|---|---|
| Functional essence core | | Omega 6 | | Omega 9 | |
| | Content | content | Content | content | Content |
| Ingredient | (mg) | percent | (mg) | percent | (mg) |
| Sacha inchi oil | 77.0 | 34.0% | 26.18 | 9.0% | 6.93 |
| Linseed oil | 215.0 | 14.2% | 30.53 | 14.1% | 30.32 |
| Algal oil | 200.0 | 1.3% | 2.60 | 18.8% | 37.60 |
| Vitamin E | 8.0 | — | — | — | — |
| Sumation | 500.0 | — | 59.31 | — | 74.85 |
| Content percent | — | — | 11.86% | — | 14.97% |

It needs to especially emphasize that, the capsule shown in FIG. 1 and FIG. 2 is not used for limiting the practical type of the vegetarian composition containing unsaturated fatty acids proposed by the present invention. It should be more flexible about the practical type of the vegetarian composition. For instance, although most of people can easily swallow a capsule type health food, it is difficult for a small number of people to smoothly swallow the capsule health food since the capsule health food may stick in esophagus. So that, in order to facilitate those small number of people can administer the vegetarian composition containing unsaturated fatty acids proposed by the present invention more easily, the aforesaid capsule housing 10 can also be re-designed to a combination type capsule.

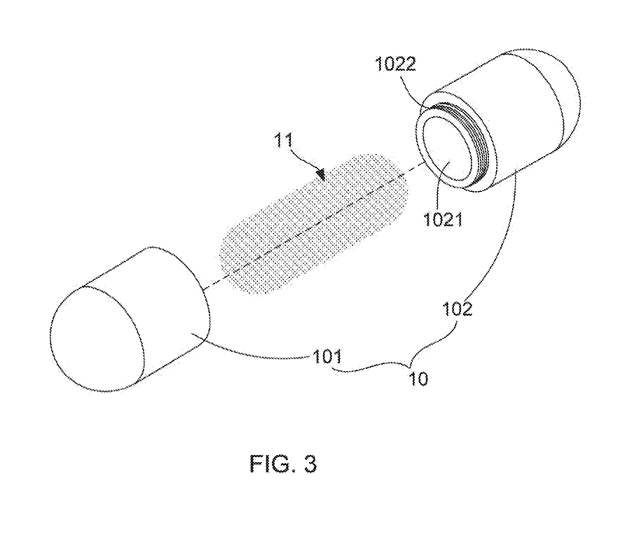

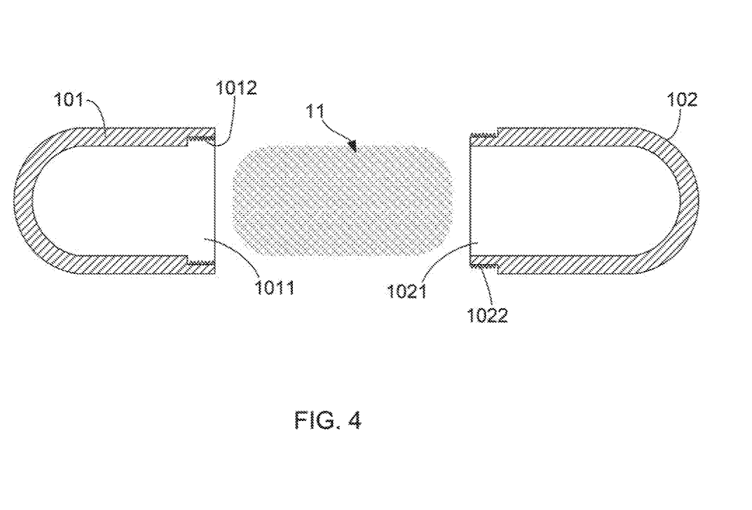

Please refer to FIG. 3, which illustrates an exploded view of a combination type capsule housing. Moreover, please simultaneously refer to FIG. 4, where a cross-sectional side view of the combination type capsule housing is provided. As FIG. 3 and FIG. 4 show, the combination type capsule housing comprises a first capsule shell 101 and a second capsule shell 102, wherein the first capsule shell 101 has a first connecting opening 1011, and the peripheral of the first connecting opening 1011 is provided with a first screw portion 1012. Opposite to the first capsule shell 101, the second capsule shell 102 has a second connecting opening 1021, and the peripheral of the second connecting opening 1021 is provided with a second screw portion 1022. By such arrangements, the first capsule shell 101 and the second capsule shell 102 can be combined to the said combination type capsule after carrying out the connection of the first connecting opening 1011 and the second connecting opening 1021 as well as the engagement between the second screw portion 1022 and the first screw portion 1012. Thus, by rotating the first capsule shell 101 or the second capsule shell 102, people can release the engagement of the second screw portion 1022 and the first screw portion 1012, and then take out the functional essence core 11 from the separated first capsule shell 101 and second capsule shell 102.

On the other hand, the functional essence core 11 is not limited to be a liquid vegetarian composition containing unsaturated fatty acids, it can also be processed to a solid vegetarian composition such as a power type functional essence core or a tablet type functional essence core. Moreover, for accommodating the power type or tablet type functional essence core, the capsule housing 10 shown in FIG. 3 and FIG. 4 can be changed to an accommodating bag or bottle.

The power type functional essence core can be fabricated by using a powder manufacturing method, wherein the manufacturing method consists of following processing steps:
step (1): mixing a Sacha inchi oil, a linseed oil, a mixture of algal oils, a vitamin E, and a vegetable emulsifier to an oil phase mixture, wherein the vegetable emulsifier is soya lecithin;
step (2): mixing adding a polysaccharide into water for obtaining an aqueous phase solution;
step (3): forming an oil-in-water emulsion by mixing the oil phase mixture and the aqueous phase solution;
step (4): treating the oil-in-water emulsion with a spray drying process, and then a power type functional essence core is produced.

Therefore, through above descriptions, the vegetarian composition containing unsaturated fatty acids provided by the present invention has been introduced completely and clearly; in summary, the present invention includes the advantages of:

(1) Differing from commercial dietary supplements of EPA and DHA are made by using abyssal fishes as primary raw materials, the present invention provides an unsaturated-fatty-acid-containing vegetarian composition, wherein the said composition is composed by at least one vegetable oil and at least one algal oil, and rich in Omega-3, Omega-6 and Omega-9. Moreover, it is worth introducing that, this vegetarian composition can be accommodated by a vegetable capsule shell, so as to become a capsule type of dietary supplement. Therefore, it is a great relief for vegetarians to administer the vegetarian composition containing unsaturated fatty acids because the vegetarian composition is made of vegan materials.

(2) On the other hand, the present invention particularly design the constituting ratio of Omega-3, Omega-6, and Omega-9 in order to facilitate the content levels of Omega-3 and Omega-6 in the body of a vegetarian administering the vegetarian composition of the present invention reach an ideal balance state.

The above description is made on embodiments of the present invention. However, the embodiments are not intended to limit scope of the present invention, and all equivalent implementations or alterations within the spirit of the present invention still fall within the scope of the present invention.

What is claimed is:

1. A vegetarian composition containing unsaturated fatty acids, comprising a vegetable oil at 50-60 weight percent, an algal oil at 35-50 weight percent and a vitamin E at below 5 weight percent based on the total weight of the vegetarian composition;
wherein the vegetable oil is a mixture of Sacha inchi oil and linseed oil;
wherein the algal oil is a mixture of an algal oil extracted from Crypthecodinium cohnii and an algal oil extracted from Schizochytrium sp.;
wherein the said unsaturated fatty acids contained by the vegetarian composition are an Omega-3 fatty acid, an Omega-6 fatty acid and an Omega-9 fatty acid; and
wherein the Omega-3 fatty acid having a first content percent of 45-55%, the Omega-6 fatty acid having a second content percent of 5-15%, and the Omega-9 fatty acid having a third content percent of 10-20%.

2. The vegetarian composition of claim 1, being a liquid vegetarian composition or a solid vegetarian composition.

3. The vegetarian composition of claim 1, wherein the Omega-3 fatty acid further comprises an α-linolenic acid (ALA), an eicosapantaenoic acid (EPA), and a docosahexaenoic acid (DHA).
